(19) United States
(12) Patent Application Publication
(10) Pub. No.: US 2017/0367679 A1
(43) Pub. Date: Dec. 28, 2017

(54) WEARABLE ULTRASONIC DEVICE FOR HEALTH MONITORING WITH DISPLAY

(71) Applicant: MiiCs & Partners (Shenzhen) Co., Ltd., Shenzhen (CN)

(72) Inventor: JUAN WANG, Shenzhen (CN)

(21) Appl. No.: 15/333,324

(22) Filed: Oct. 25, 2016

(30) Foreign Application Priority Data
Jun. 27, 2016 (CN) 201610477104.0

(51) Int. Cl. A61B 8/00; H03H 9/15; G06K 9/00; G06T 7/00; A61B 5/1172; H03H 9/205; H03H 9/13

(52) U.S. Cl. CPC A61B 8/4227; H03H 9/205; H03H 9/15; H03H 9/131; G06T 7/0012; A61B 8/4494; A61B 8/462; A61B 5/1172; G06K 9/00013; A61B 2562/164; G06T 2207/10132; G06T 2207/30004

(57) ABSTRACT

A wearable ultrasonic device constantly emitting waves attached with a readout display device includes a frame defining a space, an ultrasonic sensor, and a display panel received in the space. The ultrasonic sensor monitors a user's health. The display panel is positioned on the ultrasonic sensor. The ultrasonic sensor includes a signal transmitting layer configured to emit ultrasonic waves. The signal transmitting layer includes a second electrode layer and a plurality of piezoelectric units formed on the second electrode layer. Each piezoelectric unit includes a second piezoelectric material layer formed on the second electrode layer and a conductive layer formed on the second piezoelectric material layer.

### FIELD

The subject matter herein generally relates to a wearable ultrasonic device for health monitoring.

### BACKGROUND

Ultrasonic sensors have many advantages such as small size, cheap price, safety, and widespread use in medical devices. The ultrasonic device can be used for medical diagnosis which produces a result and transmits the result to a display device separate from the ultrasonic device. However, results obtained from the ultrasonic sensor may not be accurate when air is positioned between the ultrasonic sensor and the user's skin, and there is a desire to readily ascertain the user's health results on a display. Therefore, there is room for improvement in the art.

### BRIEF DESCRIPTION OF THE DRAWINGS

Implementations of the present technology will now be described, by way of example only, with reference to the attached figures.

FIG. 1 is a cross-sectional view of an exemplary embodiment of an ultrasonic device.

FIG. 2 is cross-sectional view of a first exemplary embodiment of an ultrasonic sensor in the ultrasonic device of FIG. 1.

FIG. 3 is cross-sectional view of a second exemplary embodiment of a signal transmitting layer.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The term “coupled” is defined as connected, whether directly or indirectly through intervening components, and is not necessarily limited to physical connections. The connection can be such that the objects are permanently connected or releasably connected. The term “comprising” when utilized, means “including, but not necessarily limited to”; it specifically indicates open-ended inclusion or membership in the so-described combination, group, series, and the like.

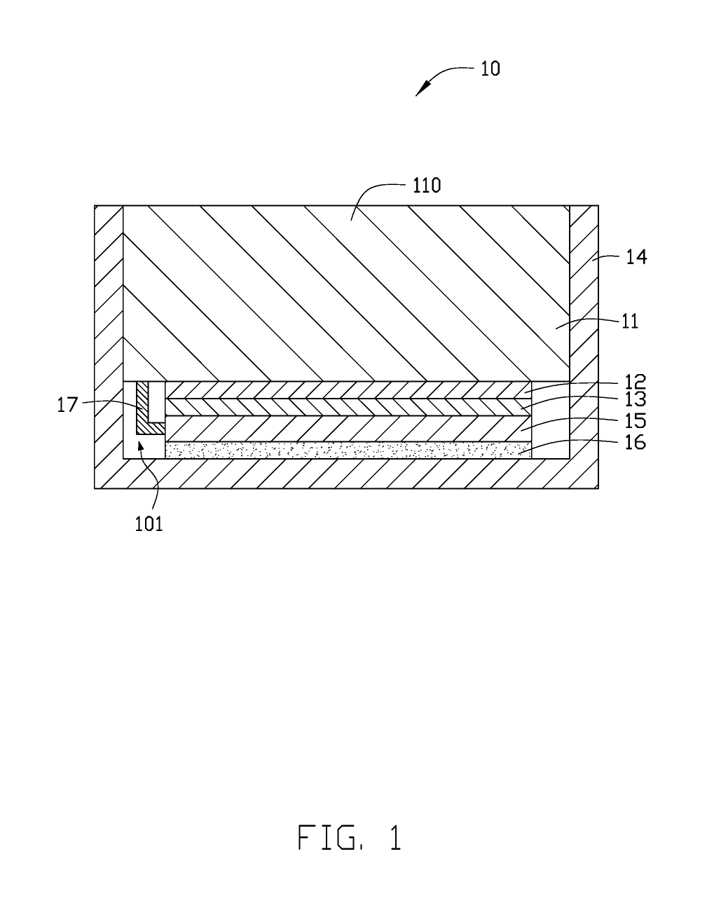

FIG. 1 illustrates a wearable ultrasonic device 10 according to an exemplary embodiment. The wearable ultrasonic device 10 is configured for medical examination and diagnosis and is able to constantly monitor a user's health characteristics such as blood flow, blood pressure, heart rate, etc.

The wearable ultrasonic device 10 includes an ultrasonic sensor 15, a shielding layer 13 positioned on the ultrasonic sensor 15, a buffer layer 12 positioned on the shielding layer 13, a display panel 11 positioned on the buffer layer 12, and a frame 14. The frame 14 defines a space 101 to receive the ultrasonic sensor 15, the shielding layer 13, the buffer layer 12, the display panel 11, and other elements not shown (for example battery and chip) of the wearable ultrasonic device 10.

The display panel 11 is mounted on the frame 14 and includes a display surface 110 to show images. The display surface 110 is exposed from the frame 14. The ultrasonic sensor 15 is located at a side of the display panel 11 facing away from the display surface 110. The ultrasonic sensor 15 is coupled to the frame 14 by a first adhesive layer 16. The first adhesive layer 16 may be flexible.

The ultrasonic sensor 15 is electrically coupled to the display panel 11 by a signal transmitting module 17. The ultrasonic sensor 15 can monitor a user's blood flow, blood pressure, and heart rate when the ultrasonic sensor 15 is placed on skin of the user and produce data signals corresponding to the monitoring. The data signals corresponding to the result of monitoring are transmitted to the display panel 11 by the signal transmitting module 17, and the display panel 11 displays the data signals as images or as information in other form on the display surface 110. The signal transmitting module 17 may be a flexible printed circuit.

The display panel 11 may be a known organic light emitting diode (OLED) display panel or a known liquid crystal display (LCD) panel. In this embodiment, the display panel 11 may be a flexible OLED display panel, so the wearable ultrasonic device 10 can be attached to the body or limb of the user. In other embodiments, the display panel 11 may be an LCD panel having a curved shape to fit any part of the user's body.

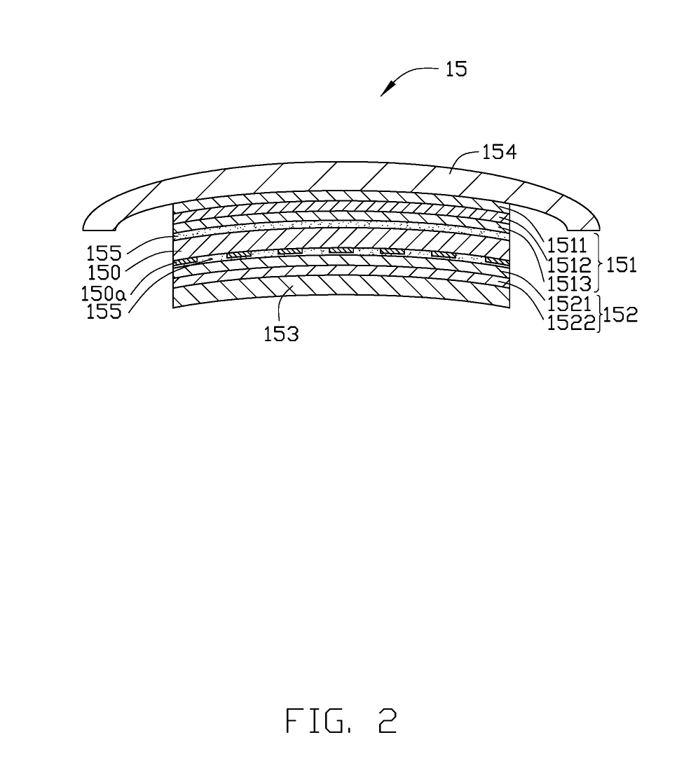

FIG. 2 illustrates the ultrasonic sensor 15 according to an exemplary embodiment. The ultrasonic sensor 15 includes a substrate 150, a signal receiving layer 152, a signal transmitting layer 151, a flexible layer 153, and a protecting layer 154. The signal receiving layer 152 is coupled to a surface of the substrate 150 by a second adhesive layer 155. The signal transmitting layer 151 is coupled to a surface of the substrate 150 facing away from the signal receiving layer 152 by another second adhesive layer 155. The signal transmitting layer 151 is closer to the display panel 11 than the signal receiving layer 152. In this exemplary embodiment, the ultrasonic sensor 15 has a curved shape (e.g. arc shape). The second adhesive layer 155 is flexible. The second adhesive layer 155 and the first adhesive layer 160 can be made of a same material or different materials.

The signal transmitting layer 151 is configured to emit ultrasonic waves. The signal receiving layer 152 is configured to receive ultrasonic waves reflected by a human body or body part to which the wearable ultrasonic device 10 is attached. The signal receiving layer 152 includes a first piezoelectric material layer 1521 and a first electrode layer 1522 positioned on the first piezoelectric material layer 1521. The first piezoelectric material layer 1521 is coupled to the substrate 150 by the second adhesive layer 155. That is, the second adhesive layer 155 is positioned between the substrate 150 and the first piezoelectric material layer 1521.

The signal transmitting layer 151 includes a second electrode layer 1511, a third electrode layer 1513, and a second piezoelectric material layer 1512 positioned between the second electrode layer 1511 and the third electrode layer 1513. The third electrode layer 1513 is coupled to the substrate 150 by the second adhesive layer 155. That is, the second adhesive layer 155 is positioned between the substrate 150 and the third electrode layer 1513.

The first piezoelectric material layer 1521 and the second piezoelectric material layer 1512 can be made of polyvinylidene fluoride (PVDF). The first electrode layer 1522, the second electrode layer 1511, and the third electrode layer 1513 can be made of a same electrically conductive material or different electrically conductive materials.

The substrate 150 can be made of a flexible material, such as polyimide or polyethylene terephthalate. In some embodiments, the substrate 150 is made of rigid material, such as glass. A plurality of thin film transistors 150a is formed on the substrate 150. The plurality of thin film transistors 150a is arranged in an array and is electrically coupled to the signal receiving layer 152 and the signal transmitting module 17. The thin film transistors 150a are configured to receive electrical signals from the signal receiving layer 152, convert the electrical signals to data signals, and transmit the data signals to the display panel 11 by the signal transmitting module 17.

The flexible layer 153 is formed on a surface of the signal receiving layer 152 facing away from the signal transmitting layer 151. The flexible layer 153 is configured to protect the signal receiving layer 152. The protecting layer 154 is formed on a surface of the signal transmitting layer 151 facing away from the signal receiving layer 152. The protecting layer 154 is configured to protect the signal transmitting layer 151. The flexible layer 153 and the protecting layer 154 are made of rubber or other common flexible material.

The shielding layer 13 covers a surface of the ultrasonic sensor 15 adjacent to the display panel 11, and particularly, covers a surface of the protecting layer 154 adjacent to the display panel 11. The shielding layer 13 is made of an electrically conductive material and is configured to avoid any electrical activity in the display panel 11 interfering with the ultrasonic sensor 15. In addition, the shielding layer 13 is flexible. In some embodiments, the shielding layer 13 may cover at least two surfaces of the ultrasonic sensor 15. The ultrasonic sensor 15 being substantially enclosed by the shielding layer 13.

The buffer layer 12 is positioned between the shielding layer 13 and the display panel 11. The buffer layer 12 is elastic and configured to protect the ultrasonic sensor 15 against shock and impact. For example, the buffer layer 12 can be made of an elastic rubber. In some embodiments, the buffer layer 12 can be omitted.

In this embodiment, the frame 14 can be made of a flexible material, such as polyimide or polyethylene terephthalate. In some embodiments, the frame 14 is made of a rigid material. In some embodiments, the frame 14 may be made of a rigid material. Moreover, the frame 15 may have a curved shape to fit any part of the user's body in a snug fashion.

As an example of when in use, the wearable ultrasonic device 10 is attached to a user's skin by attaching the frame 14 on the user's skin. A voltage is applied between the second electrode layer 1511 and the third electrode layer 1513, and the second piezoelectric material layer 1512 vibrates and emits ultrasonic waves. The ultrasonic waves pass through the user's skin to the subcutaneous fatty tissue, and a portion of the ultrasonic waves is reflected by the subcutaneous fatty tissue to the signal receiving layer 152. The reflected ultrasonic waves change according to the status of the subcutaneous fatty tissue. The signal receiving layer 152 converts the received ultrasonic wave signals to electrical signals and transmits the electrical signals to the thin film transistors 150a. The thin film transistors 150a convert the electrical signals to data signals, and transmit the data signals to the display panel 11. The user can observe the images or other information on the display panel 11.

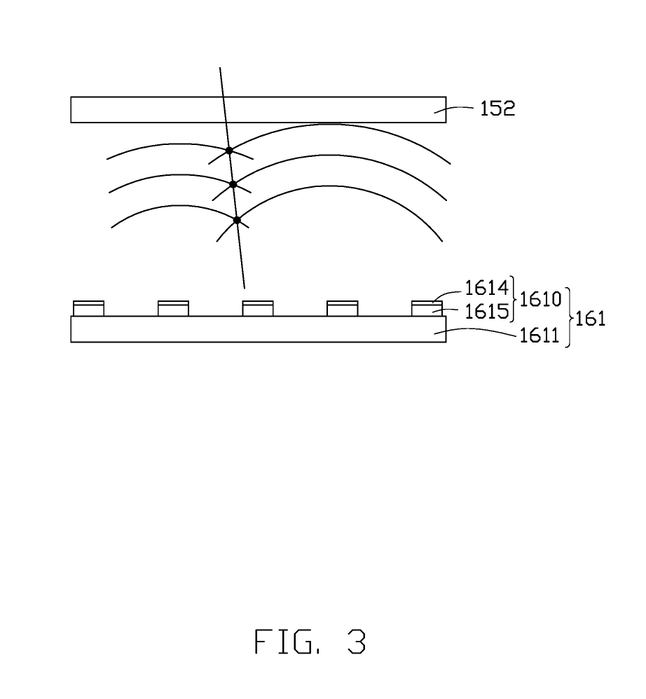

FIG. 3 illustrates a signal transmitting layer 161 according to a second exemplary embodiment. The signal transmitting layer 161 can be used in the ultrasonic sensor 15 and can be substituted for the signal transmitting layer 151.

The signal transmitting layer 161 is configured to emit ultrasonic waves continuously. The signal transmitting layer 161 includes a second electrode layer 1611 and a plurality of piezoelectric units 1610 formed on the second electrode layer 1611, wherein the piezoelectric units 1610 are closer to the substrate 21 than the second electrode layer 1611. The piezoelectric units 1610 are separate from each other. Each piezoelectric unit 1610 includes a second piezoelectric material layer 1615 formed on the second electrode layer 1611 and a conductive layer 1614 formed on the second piezoelectric material layer 1615 facing away from the second electrode layer 1611. Each second piezoelectric material layer 1615 is able to vibrate and emit ultrasonic waves when a voltage is applied between the second electrode layer 1611 and the corresponding conductive layer 1614.

Each piezoelectric unit 1610 can emit ultrasonic waves independently called “beam forming mode”. In the beam forming mode, ultrasonic waves emitted from one piezoelectric unit 1610 overlap with ultrasonic waves emitted from other piezoelectric unit 1610, as such intensity of the ultrasonic waves from the signal transmitting layer 161 can be effectively improved. The piezoelectric units 1610 can emit ultrasonic waves at a same time or at different times, for example the piezoelectric units 1610 emit ultrasonic waves in certain order. The piezoelectric units 1610 can emit ultrasonic waves having a same intensity or different intensities.

The first piezoelectric material layer 1521 and the second piezoelectric material layer 1615 can be made of polyvinylidene fluoride (PVDF). The first electrode layer 1522, the second electrode layer 1611, and the conductive layer 1614 can be made of a same electrically conductive material or different electrically conductive materials.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A wearable ultrasonic device comprising:
a frame, the frame defining a space;
an ultrasonic sensor received in the space and configured to monitor a user's health status;
a display panel positioned on the ultrasonic sensor and received in the space, the display panel being electrically coupled to the ultrasonic sensor and configured to display monitoring results from the ultrasonic sensor;
wherein the ultrasonic sensor comprises a substrate, a signal receiving layer, and a signal transmitting layer positioned on the substrate, wherein the signal transmitting layer is configured to emit ultrasonic waves, and wherein the signal transmitting layer comprises a second electrode layer and a plurality of piezoelectric units spaced apart from each other and formed on the second electrode layer; and
wherein each piezoelectric unit comprises a second piezoelectric material layer formed on the second electrode layer and a conductive layer formed on the second piezoelectric material layer.

2. The wearable ultrasonic device of claim 1, wherein the signal receiving layer comprises a first piezoelectric material layer and a first electrode layer positioned on the first piezoelectric material layer.

3. The wearable ultrasonic device of claim 2, wherein a plurality of thin film transistors is arranged in an array and formed on the substrate, the plurality of thin film transistors is electrically coupled to the signal receiving layer and is configured to receive electrical signals from the signal receiving layer.

4. The fingerprint identification device of claim 1, wherein the signal receiving layer is coupled to a surface of the substrate by a second adhesive layer; the signal transmitting layer is coupled to a surface of the substrate facing away from the signal receiving layer by another second adhesive layer; the signal transmitting layer is adjacent to the display panel.

5. The wearable ultrasonic device of claim 4, wherein the second adhesive layers are flexible.

6. The wearable ultrasonic device of claim 1, wherein the ultrasonic sensor is coupled to the frame by a first adhesive layer.

7. The wearable ultrasonic device of claim 6, wherein the first adhesive layer is flexible.

8. The wearable ultrasonic device of claim 1, wherein the frame is made of a flexible material.

9. The wearable ultrasonic device of claim 1, wherein the frame is made of a rigid material.

10. The wearable ultrasonic device of claim 9, wherein the frame has a curved shape to fit the user's body.

11. The wearable ultrasonic device of claim 1, further comprising a shielding layer positioned between the ultrasonic sensor and the display panel; the shielding layer covers at least one surface of the ultrasonic sensor; the shielding layer is made of an electrically conductive material.

12. The wearable ultrasonic device of claim 1, further comprising a buffer layer positioned between the ultrasonic sensor and the display panel; wherein the buffer layer is elastic.
